(19) United States
(12) Patent Application Publication
(10) Pub. No.: US 2022/0326255 A1
(43) Pub. Date: Oct. 13, 2022

(54) METHODS OF MONITORING MUCOSAL HEALING

(71) Applicant: MediBeacon Inc., St. Louis, MO (US)

(72) Inventors: Phillip I. Tarr, Clayton, MO (US); Richard B. Dorshow, St. Louis, MO (US)

(21) Appl. No.: 17/708,789

(22) Filed: Mar. 30, 2022

Related U.S. Application Data
(60) Provisional application No. 63/169,568, filed on Apr. 1, 2021.

(51) Int. Cl. G01N 33/68; A61B 5/00; A61K 31/4965; G01N 33/493

(52) U.S. Cl. CPC G01N 33/6893; A61B 5/0071; A61B 5/4238; A61K 31/4965; G01N 33/493; G01N 2800/065

(57) ABSTRACT

The disclosure provides for methods for monitoring mucosal healing in a patient with a digestive disease, or for use in a pre-disease state, and includes intestinal as well as extra-intestinal disorders in which gut permeability is increased. The method may include establishing a baseline of the patient, treating the patient for the digestive disease or the pre-disease state, measuring gut permeability of the patient after treatment, and comparing a second total percentage of the administered dose recovered to the baseline total percentage of the administered dose recovered. Establishing the baseline may include enterally administering a first dosage of a composition comprising a fluorescent tracer, measuring a first amount of the administered dose that can be found outside the gut over a period of time, and determining a baseline total percentage of the administered dose recovered. Measuring gut permeability may include enterally administering a second dosage of the composition, measuring a second amount of the administered dose, and determining a second total percentage of the administered dose recovered.

### CROSS-REFERENCE TO RELATED APPLICATIONS

This application claims priority to U.S. Provisional Application No. 63/169,568, filed Apr. 1, 2021, the contents of which are entirely incorporated by reference herein.

### FIELD

The present disclosure relates generally to methods of monitoring intestinal mucosal healing in a patient.

### BACKGROUND

In the healthy state, the intestinal barrier permits selective translocation (absorption) of water, electrolytes, and nutrients from the gut lumen into the systemic circulation, while preventing entry of undigested food, macromolecules, and microorganisms into the body. Increased gut permeability infers loss of barrier integrity, principally through disruption of tight junctions between epithelial cells. Gut permeability is increased in a variety of intestinal inflammatory conditions, including Crohn's Disease, graft versus host disease, and celiac disease.

Endoscopy has been the primary method to evaluate mucosal healing. Endoscopy enables the gastroenterologist to visually examine the mucosa and to obtain biopsies that can be examined microscopically for inflammation or other signs of tissue damage. However, patients with inflammatory bowel diseases (IBD) undergo many endoscopies, which can be a source of dissatisfaction, particularly because of the bowel prep. Endoscopic monitoring is also expensive (including time off from work), and, on occasion, can cause intestinal perforation and hemorrhage.

The typical measurement of intestinal permeability involves oral ingestion of probe molecules, which are not metabolized in the gut or circulation, and which are excreted in urine where they can be measured. The most widely use test is the dual sugar absorption test (DSAT), and involves an oral challenge with a disaccharide (usually lactulose) and a monosaccharide (mannitol or, increasingly, rhamnose) and assays of urine to determine the extent to which the sugars are absorbed by the host. While the DSAT is considered state of the art for assessing gut permeability, several limitations diminish its utility. Most of these are technical limitations related to specimen timing, handling, and assay variability, each of which reduces the theoretical appeal of the DSAT.

Thus, there remains a need for improved methods for monitoring mucosal healing and management of patients with digestive diseases and other conditions involving increased intestinal permeability, while reducing the need for repeated endoscopy.

### BRIEF SUMMARY

Principles and embodiments of the present disclosure relate generally to methods of monitoring mucosal healing in a patient with a digestive disease or in a pre-disease state. The method may include establishing a baseline of the patient, treating the patient for the digestive disease or the pre-disease state, measuring gut permeability of the patient after treatment, and comparing a second total percentage of the administered dose recovered to a baseline total percentage of the administered dose recovered.

Establishing the baseline of the patient may include enterally administering a first dosage of a composition comprising a fluorescent tracer that is not substantially absorbed by a healthy gut, measuring, via fluorescence, a first amount of the administered dose that can be found outside the gut over a period of time, and determining the baseline total percentage of the administered dose recovered. Measuring gut permeability of the patient after treatment may include enterally administering a second dosage of the composition comprising the fluorescent tracer, measuring, via fluorescence, a second amount of the enterally administered dose that can be found outside the gut over a period of time, and determining the second total percentage of the administered dose recovered.

Other aspects of the present disclosure relate to methods for determining mucosal healing in a patient with a digestive disease. The method may include enterally administering a dosage of a composition comprising a fluorescent tracer that is not substantially absorbed by a healthy gut, measuring, via fluorescence, an amount of the administered dose that can be found outside the gut over a period of time, and determining a total percentage of the administered dose recovered. In some aspects, the total percentage of the administered dose recovered correlates inversely to the patient's mucosal healing, i.e., the less absorbed, the less excreted or measured via the skin, the greater the barrier integrity.

Additional embodiments and features are set forth in part in the description that follows, and will become apparent to those skilled in the art upon examination of the specification or may be learned by the practice of the disclosed subject matter.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

The description will be more fully understood with reference to the following figures and data graphs, which are presented as various embodiments of the disclosure and should not be construed as a complete recitation of the scope of the disclosure, wherein:

FIG. 1 shows the ratio of the concentrations of MB-404 to MB-301 in urines of two rats with indomethacin-induced bowel injury (red and blue lines) and two control rats (green and black lines) at various time points following enteral fluorophore delivery.

FIG. 2 shows transdermal detection of MB-301 and MB-404 using dual-wavelength transdermal fluorescence detection to monitor fluorophore concentration in two rats with indomethacin-induced bowel injury (red and blue lines) and two control rats (green and black lines). Shown are the area under the curves (AUC) of MB-301 (dashed lines) and MB-404 (solid lines) fluorescence over time.

FIG. 3A and FIG. 3B show transdermal fluorescence detection of MB-404 concentration in two rats with indomethacin-induced bowel injury (red and blue lines) and two control rats (green and black lines) in two separate experiments.

FIG. 4A shows MB-102 excretion after MB-102 was delivered to two rats with indomethacin-induced bowel injury (blue and orange lines) and two control rats (grey and yellow lines). Shown is the % Administered Dose Recovered in urine at various time points post-administration.

FIG. 4B shows transdermal fluorescence detection of MB-102 concentration in three rats with indomethacin-induced bowel injury (red, black and green lines) and four control rats (purple, pink, turquoise and yellow lines).

FIG. 5 shows the percent of the administered dose of lactulose or MB-102 recovered in control research participants and research participants with Crohn's disease.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The terms “comprising” or “having” mean “including, but not necessarily limited to”; specifically indicate open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention. The terms “a,” “an,” and “the” are understood to encompass the plural as well as the singular.

As used herein, the term “mucosal healing” is defined as a meaningful decrease in intestinal permeability compared to a baseline permeability measurement and approaching that of an average permeability measurement of a normal population. The baseline permeability measurement may be made at the time of diagnosis of a digestive disease before treatment, determination of a pre-disease state, during an exacerbation of symptoms, or after treatment has been partly completed.

As used herein, the term “meaningful” (e.g. meaningful decrease in intestinal permeability) is used to describe a sufficient magnitude to indicate an informative clinical effect or non-effect (and support a decision for dose/treatment escalation, de-escalation, or holding steady).

As used herein, the terms “gut”, “intestine”, and “GI” are used to describe the hollow organs of the gastrointestinal tract of the patient, in particular the stomach, small intestine, large intestine, and esophagus. As used herein, the term “healthy gut” may be defined by a physician and may be a gut in a patient without a digestive disease and not in a pre-disease state.

As used herein, the term “recovered” refers to a measure of the amount of tracer in plasma, urine, circulating blood, tissue, or any location of the body outside the lumen of the gut. For example, a total percentage of an administered dose of a tracer that is recovered may be a direct measurement of the amount of tracer in a body fluid sample or may be an indirect measurement of the amount of tracer outside the gut through transcutaneous measurement of the tracer.

As used herein, the term “normal population” may be defined by a physician and may include two or more patients without a digestive disease and not in a pre-disease state. In an example, a normal population range may be two standard deviations above/below the mean of the reference normal population. Standard deviations and percentiles may be subject to the normality of the distribution of the normal population values and may be subject to variations based on age, sex, and/or race.

As used herein, the term “substantially” may refer to up to about 95%, up to about 98%, up to about 99%, up to about 99.5%, or up to about 99.9%. For example, the phrase “fluorescent tracer not substantially absorbed by a healthy gut” may refer to a fluorescent tracer that is less than 5%, less than 2%, less than 1%, less than 0.5%, or less than 0.1% absorbed by a healthy gut.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

The principles and embodiments of the present disclosure relate to methods of monitoring mucosal healing, GI inflammation, and/or treatment efficacy in a patient. Accordingly, various embodiments of the present disclosure provide methods of monitoring mucosal healing, GI inflammation, and/or treatment efficacy a patient with a digestive disease or in a pre-disease state.

Fluorescent tracer agent-based monitoring systems may provide measured gastrointestinal (GI) permeability quantification in real-time and at the point of care in the management of digestive diseases or conditions associated with increased intestinal permeability. Non-limiting examples of digestive diseases or extra-intestinal conditions associated with increased intestinal permeability include IBDs consisting broadly of Crohn's Disease and ulcerative colitis, graft-versus-host disease of the intestines, nonalcoholic fatty liver disease, celiac disease, and eosinophilic esophagitis as well as type 1 or type 2 diabetes. Patients may be monitored while undergoing treatment to determine the extent of intestinal permeability and decreases in permeability (or mucosal healing) as treatment progresses. In Crohn's disease and ulcerative colitis, mucosal healing is linked to the most important patient outcomes, namely, avoidance of surgery and hospitalization, and maintenance of a high quality of life. For these IBDs, mucosal healing may include healing of mucosal ulcerations. In some embodiments, the patient may be in a pre-symptomatic or a pre-disease state. For example, the patient may be pre-IBD, pre-diabetic, or pre-fatty liver disease, or pre-fibrotic liver disease (an extension of fatty liver disease) by virtue of genetic or metabolic risk factors. Increased permeability can precede overt gastrointestinal disease (e.g. IBD) or extra-intestinal disease (e.g. type 1 diabetes). In these situations, increased intestinal permeability may be used as a marker of progression of the process leading to the disease, and prompt action, and then following the effect of the action by testing for restored barrier function (i.e. mucosal healing).

Monitoring mucosal healing during treatment provides great benefit to the patient and health care system as biologics are often used as the major treatment of these conditions. Biologics are expensive and can have harmful side effects with long term treatment. Early detection of mucosal healing can provide the clinician the ability to reduce costs in treatment and reduce side effects from the primary medication by giving them the ability to reduce the dose, or prolong the interval between dosing.

In an embodiment, a method for monitoring mucosal healing, GI inflammation, and/or treatment efficacy in a patient with a digestive disease may include establishing a baseline of the patient, treating the patient for the digestive disease, and measuring gut permeability of the patient after treatment.

The baseline may be patient specific, such that the baseline may be established at the time of diagnosis before treatment, or during an exacerbation of symptoms. In some embodiments, establishing the baseline of the patient may include enterally administering a first dosage of a composition comprising a fluorescent tracer that is not substantially absorbed by a healthy gut, measuring, via fluorescence, a first amount of the administered dose that can be found outside the gut over a period of time, and determining a baseline total percentage of the administered dose recovered. The appearance and quantification of the tracer agent outside the gut indicates the gut has increased permeability. The baseline may be used to compare with measurements made after treatment of the patient commences.

In the treatment of the patient, a clinician may prescribe a patient with a medication for the treatment of a digestive disease or an increased intestinal permeability condition may prescribe other interventions that have a direct or indirect effect on gut permeability. These medications and/or interventions include, but are not limited to, dietary manipulations, probiotics, other microbial management strategies, small molecule therapeutics, repurposed drugs, drugs that induce immune tolerance, antibiotics, intravenous immune globulin, latiglutenase or other drugs that degrade dietary gluten, and/or drugs that prevent tissue transglutaminase from modifying gluten. In at least one example, the treatment may include a medication regimen. Non-limiting examples of medications for the treatment of the digestive disease include anti-inflammatory drugs, antibiotics, immune system suppressors, Janus kinase inhibitors, and biologics. Anti-inflammatory drugs may include, but are not limited to corticosteroids such as prednisone, prednisolone, methylprednisolone, and budesonide; and orally-administered 5-aminosalicylates such as sulfasalazine, balsalazide, olsalazine and mesalamine. Antibiotics may include, but are not limited to, ciprofloxacin and metronidazole. Immune system suppressors may be used alone or in combination and include, but are not limited to azathioprine, cyclosporine, 6-mercaptopurine, tacrolimus, and methotrexate. Janus kinase inhibitors such as tofacitinib may be used to treat moderate to severe ulcerative colitis. Types of biologics used to treat Crohn's disease include, but are not limited to natalizumab, vedolizumab, infliximab, adalimumab, certolizumab pegol, golimumab, and ustekinumab. Other treatments may include the strategy known as treat-to-target, which involves treating the target of inflammation until there is both symptomatic and endoscopic and microscopic improvement or resolution.

In some embodiments, measuring gut permeability of the patient after treatment may include enterally administering a second dosage of the composition comprising the fluorescent tracer, measuring, via fluorescence, a second amount of the administered dose that can be found outside the gut over a period of time, and determining a second total percentage of the administered dose recovered. After the patient begins the treatment, changes in intestinal permeability may be measured at different time points during the treatment to determine efficacy of the medication, dosing, and regimen. The mucosal healing may be assessed after treatment for at least 5 days, at least 1 week, at least 2 weeks, at least 3 weeks, at least 1 month, at least 3 months, and/or at least 6 months. Subsequent assessments may be done on a weekly or bi-weekly basis to determine whether the medication and the regimen has impacted mucosal healing.

In an embodiment, the method may further include comparing the second total percentage of the administered dose recovered to the baseline total percentage of the administered dose recovered. In some embodiments, the method may additionally include assessing the patient's mucosal healing based on the comparison of the second total percentage of the administered dose recovered to the baseline total percentage of the administered dose recovered. A decrease in fluorescence measured from baseline to a specified time point after the treatment begins indicates there may be mucosal healing and that the medicine and/or treatment regimen may be effective. The dose of the medication for treatment may be adjusted at this point, the regimen may be modified, or the medication and/or regimen may be maintained. No change or an increase in fluorescence measured from baseline to a specified time point after the treatment begins indicates the lack of mucosal healing and that the medication or dosing regimen needs to be reassessed. In some embodiments, the patient's mucosal healing may be assessed more than one time. At the height of illness (e.g., a flare, for people known to have IBD, or the baseline at the time of diagnosis for newly identified patients), a meaningful decrease in the fluorescence value may be viewed as an improvement in the mucosal healing.

In an example, if the second total percentage of the administered dose recovered is less than or equal to 40%, 50%, or 60% of the baseline total percentage of the administered dose recovered, the patient likely has mucosal healing and the treatment may be continued, reduced, or stopped. Stopping treatment may include slowly weaning the patient off the medication. In another example, if the second total percentage of the administered dose recovered is greater than 60%, 50%, or 40% of the baseline total percentage of the administered dose recovered, the patient likely does not have mucosal healing, and, in fact, additional intervention may be needed. In some examples, failure to meaningfully reduce the permeability from the baseline determination, (e.g. no or slight change from baseline, assuming baseline is determined while the patient is in a highly unhealed state), may prompt an intervention, an escalation, or at least a reevaluation.

The intervention may include discontinuing the treatment, optimizing the treatment, or providing one or more subsequent treatments. For example, the intervention may include replacing the first medication with a second medication, increasing or decreasing the dose of the initial treatment, or adjusting the timing for when the dose is provided. The mucosal healing of the patient may then be assessed after the intervention. In some examples, the intervention which may lead to higher rates of mucosal healing, deep remission, and reduced prednisone use compared with patients without treatment or mucosal healing.

In another example, the second total percentage of the administered dose recovered may be compared to an average total percentage of the administered dose recovered in a normal population. If the second total percentage of the administered dose recovered is less than the baseline total percentage of the administered dose recovered and less than or equal to the 95th percentile of an average total percentage of the administered dose recovered in a normal population, then the patient may have mucosal healing. In this case, the indication of mucosal healing may result in not escalating treatment, or even slowly diminishing treatment. In an additional example, if the second total percentage of the administered dose recovered is at or near a personal baseline established prior to an exacerbation of symptoms, the patient may have mucosal healing. Alternatively, if the second total percentage of the administered dose recovered is less than the baseline total percentage of the administered dose recovered but above the 95th percentile of an average total percentage of the administered dose recovered in a normal population, then the patient may not have mucosal healing and it may be an indication to be more aggressive with the treatment, trying to achieve a population based normal range. If further treatment does not further reduce the value, that might be acceptable, and then the second total percentage of the administered dose recovered may be the patient's provisional baseline, and further monitoring may be used to confirm no deterioration in mucosal status as compared to the provisional baseline. In one example, a well-controlled patient with colonoscopic evidence of mucosal healing may not return to the 95th percentile of an average total percentage of the administered dose recovered, but the side effects of being more aggressive negate the theoretical value of complete mucosal healing. In this case, the treatment may not be escalated, or even may be slowly diminished.

In another embodiment, a method for determining mucosal healing in a patient with a digestive disease may include enterally administering a dosage of a composition comprising a fluorescent tracer that is not substantially absorbed by a healthy gut, measuring, via fluorescence, an amount of the administered dose that can be found outside the gut over a period of time, and determining a total percentage of the administered dose recovered based on the amount measured. In some embodiments, the total percentage of the administered dose recovered may correlate to the patient's mucosal healing. The amount of the administered dose that can be found outside the gut may be measured in the patient's blood, tissue, or urine, either directly or transcutaneously.

In some embodiments, the method may further include assessing the patient's mucosal healing. The patient likely has mucosal healing if the total percentage of the administered dose recovered or measured outside the gut is less than or equal to about 0.5%, 1%, or 1.5% or the measure of fluorescence correlates to less than or equal to about 0.5%, 1%, or 1.5% of the administered dose. The patient likely does not have mucosal healing if the total percentage of the administered dose recovered is greater than about 1.5%, 2%, or 2.5% or the measure of fluorescence correlates to greater than about 1.5%, 2%, or 2.5% of the administered dose recovered. The percentage of the administered dose recovered may be assessed over at least 6 hours, at least 12 hours, at least 24 hours, or at least 48 hours. The mucosal healing may be assessed at least daily to at least weekly for a period of at least 5 days, at least 1 week, at least 2 weeks, at least 3 weeks, at least 1 month, at least 3 months, and/or at least 6 months. The mucosal healing may be measured one or more times within the assessment period.

In an embodiment, the patient's mucosal healing may be assessed by comparing the total percentage of the administered dose recovered or measured outside the gut in the patient to a total percentage of the administered dose recovered in a patient with a healthy gut. In some embodiments, the total percentage of the administered dose recovered or measured may be compared to a distribution of values of the administered dose recovered in a normal population. The dose administered to the patient may be the same or a similar dose administered to the normal population. Therefore, the percentage of the administered dose that is recovered or measured may be compared between the two groups. If the total percentage of the administered dose recovered or measured in the patient is less than or equal to about 100%, 150%, 200%, or more of an average total percentage of the administered dose recovered or measured in a normal population, the patient likely has mucosal healing. In an example, if the total percentage of the administered dose recovered or measured is less than or equal to the 95th percentile of an average total percentage of the administered dose recovered or measured in a normal population, the patient likely has mucosal healing. In another example, if the total percentage of the administered dose recovered or measured is less than or equal to two standard deviations from the mean of the administered dose recovered or measured in a normal population, the patient likely has mucosal healing. If the total percentage of the administered dose recovered or measured in the patient is greater than about 200%, 150%, or 100% of an average total percentage of the administered dose recovered or measured outside the gut in a normal population, the patient likely does not have mucosal healing. In an example, if the total percentage of the administered dose recovered or measured is greater than the 95th percentile of an average total percentage of the administered dose recovered in a normal population, the patient likely does not have mucosal healing. In an example, if the total percentage of the administered dose recovered is greater than two standard deviations above the mean of the administered dose recovered in a normal population, the patient likely does not have mucosal healing. In another example, total percentage of the administered dose recovered in patients with a digestive disease may be 2-2.5 fold higher than the signal in a normal population.

In some embodiments, a method for determining mucosal healing in a patient with a digestive disease may include enterally administering a dosage of a composition comprising a first fluorescent tracer that is not substantially absorbed by a healthy gut, administering a second fluorescent tracer, measuring, via fluorescence, an amount of the administered first fluorescent tracer and second fluorescent tracer that can be found outside the gut over a period of time, and determining the patient's mucosal healing by a ratio of the concentration of the first fluorescent tracer to a concentration of the second fluorescent tracer in the patient's blood, tissue, or urine, either directly or transcutaneously, allowing distinction between healthy and diseased intestinal mucosa.

The ratio of the concentration of the first fluorescent tracer to the concentration of the second fluorescent tracer may be predictive of the patient's mucosal healing. For example, the ratio may be improved after treatment of a digestive disease or another condition in which there is increased intestinal permeability in the patient. The ratio in the untreated state may be 2-3 times the upper limit of the ratio in a normal population (e.g. normal range). In some embodiments, treatment of the disease or condition may reduce the ratio to within the normal range. In various embodiments, the fluorophores may have similar molecular weights to mannitol, rhamnose, and/or lactulose. For example, the first fluorescent tracer may have a molecular weight similar to lactulose and the second fluorescent tracer may have a molecular weight similar to rhamnose and/or mannitol. In one example, a ratio in the range of about 0.07-0.10 may be indicative of inflammation in Crohn's disease and a normal range may be about 0.05 to 0.02.

In some examples, the assessment of the patient's mucosal healing may be further supported by imaging or biopsies taken through an endoscope or colonoscope. These assessments may be performed less frequently due to the use and measurement of the fluorescent tracer.

In each embodiment, the fluorescent tracer agent may be administered in a single dose or in multiple doses. Doses may vary depending upon, for example, the particular fluorescent tracer agent employed or the method of detection. For example, the dosage of the tracer agent may range from about 0.1 mg/kg body weight to about 35 mg/kg body weight, from about 1.5 to about 50 mg/kg body weight, from about 0.5 to about 5 mg/kg body weight, from about 1.5 to about 3 mg/kg body weight, from about 5 to about 10 mg/kg body weight, from about 10 to about 15 mg/kg body weight, from about 15 to about 20 mg/kg body weight, from about 20 to about 25 mg/kg body weight, from about 25 to about 30 mg/kg body weight, from about 30 to about 35 mg/kg body weight, from about 35 to about 40 mg/kg body weight, from about 40 to about 45 mg/kg body weight, or from about 45 to about 50 mg/kg body weight. In various examples, the first dosage and/or the second dosage may be about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 20 mg/kg, about 25 mg/mg, about 30 mg/kg, about 35 mg/kg body weight, about 40 mg/kg body weight, about 45 mg/kg body weight, or about 50 mg/kg body weight.

The fluorescent tracer may be soluble at physiological pH, not bind proteins in the blood, not be metabolized, be freely filtered at the glomerulus kidneys, not absorbed by the kidney tubules, and/or resist degradation or denaturation by stomach acid or bile-containing fluids. The fluorescent tracer may have a molecular weight between about 340 Da and 2,500 Da. For example, the fluorescent tracer may have a molecular weight of about 340 Da to about 380 Da, about 350 Da to about 400 Da, about 375 Da to about 450 Da, about 400 Da to about 500 Da, about 450 Da to about 550 Da, about 500 Da to about 1,000 Da, or about 1,500 Da to about 2,500 Da. In an example, the fluorescent tracer may have a molecular weight of 372 Da. The fluorescent tracer may have absorption, excitation, and emission wavelengths that are about 350 nm or greater. In some embodiments, the fluorescent tracer may have an absorption wavelength of about 400 nm to about 500 nm and have an emission wavelength of about 540 nm to about 620 nm. In an example, the fluorescent tracer may have an absorption wavelength of about 445 nm and an emission wavelength of about 560 nm.

The fluorescent tracer may include, but is not limited to acridines, acridones, anthracenes, anthracylines, anthraquinones, aza-azulenes, azo-azulenes, benzenes, benzimidazoles, benzofurans, benzoindocarbocyanines, benzoindoles, benzothiophenes, carbazoles, coumarins, cyanines, dibenzofurans, dibenzothiophenes, dipyrrolo dyes, flavones, fluoresceins, imidazoles, indocarbocyanines, indocyanines, indoles, isoindoles, isoquinolines, naphthacenediones, naphthalenes, naphthoquinones, phenanthrenes, phenanthridines, phenanthridines, phenoselenazines, phenothiazines, phenoxazines, phenylxanthenes, polyfluorobenzenes, purines, pyrazines, pyrazoles, pyridines, pyrimidones, pyrroles, quinolines, quinolones, rhodamines, squaraines, tetracenes, thiophenes, triphenyl methane dyes, xanthenes, xanthones, and derivatives thereof. In an embodiment, the fluorescent tracer may include a pyrazine.

The pyrazine may be a compound of Formula I,

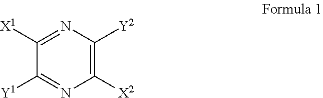

each of X¹ and X² is independently —CO₂R¹, —CONR¹R², —CO(AA) or —CONH(PS); each of Y¹ and Y² is independently selected from the group consisting of —NR¹R² and

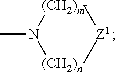

Z¹ is a single bond, —CR¹R²—, —O—, —NR¹—, —NCOR¹—, —S—, —SO—, or —SO₂—; each of R¹ to R² are independently selected from the group consisting of H, —CH₂(CHOH)ₐH, —CH₂(CHOH)ₐCH₃, —CH₂(CHOH)ₐCH₂OH, —CH₂(CHOH)ₐCO₂H, —(CHCO₂H)ₐCO₂H, —(CH₂CH₂O)_{c}H, —(CH₂CH₂O)_{c}CH₃, —(CH₂)ₐSO₃H, —(CH₂)ₐSO₃, —(CH₂)ₐSO₂H, —(CH₂)ₐSO₂⁻, —(CH₂)ₐNHSO₃H, —(CH₂)ₐNHSO₃⁻, —(CH₂)ₐNHSO₂H, —(CH₂)ₐNHSO₂⁻,—(CH₂)ₐPO₄H₃, —(CH₂)ₐPO₄H₂⁻, —(CH₂)ₐPO₄H²⁻, —(CH₂)ₐPO₄³⁻, —(CH₂)ₐPO₃H₂, —(CH₂)ₐPO₃H⁻, and —(CH₂)ₐPO₃²⁻; AA is a peptide chain comprising one or more amino acids selected from the group consisting of natural and unnatural amino acids, linked together by peptide or amide bonds and each instance of AA may be the same or different than each other instance; PS is a sulfated or non-sulfated polysaccharide chain comprising one or more monosaccharide units connected by glycosidic linkages; ‘a’ is a number from 1 to 10, ‘c’ is a number from 1 to 100, and each of ‘m’ and ‘n’ are independently a number from 1 to 3.

In some embodiments, at least one of X₁ and X₂ is —CO(PS) or —CO(AA). In yet another embodiment, both X₁ and X₂ are —CO(AA). In some embodiments, (AA) is a single amino acid selected from the group consisting of the 21 essential amino acids. In other aspects, AA is selected from the group consisting of D-arginine, D-asparagine, D-aspartic acid, D-glutamic acid, D-glutamine, D-histidine, D-homoserine, D-lysine, and D-serine. Preferably, AA is D-aspartic acid, D-glycine, D-serine, or D-tyrosine. Most preferably, AA is D-serine.

Table 1 provides a non-limiting list of exemplary fluorescent tracer agents. In at least one example, the fluorescent tracer agent may be 3,6-diamino-2,5-bis{N-[(1R)-1-carboxy-2-hydroxyethyl]carbamoyl}pyrazine or 3,6-diamino-N2,N5-bis(D-serine)-pyrazine-2,5-dicarboxamide. In another example, the pyrazine may be N²,N⁵-bis(2,3-dihydroxypropyl)-3,6-bis[(S)-2,3-dihydroxypropylamino]pyrazine-2,5-dicarboxamide. In another example, the fluorescent tracer agent may be 3,6-diamino-N²,N⁵-bis((2R,3S,4S,5S)-2,3,4,5,6-pentahydroxyhexyl)pyrazine-2-5-dicarboxamide. In another example, the fluorescent tracer agent may be 3,6-diamino-N²,N⁵-di(2,5,8,11,14,17,20,23,26,29,32,35,38,41,44,47,50,53,56,59,62,65,68-tricosaoxaheptacontan-70-yl)pyrazine-2,5-dicarboxamide. In yet another example, the fluorescent tracer agent may be (2R,2′R)-2,2′-((3,6-bis(((S)-2,3-dihydroxypropyl)amino)pyrazine-2,5-dicarbonyl)bis(azanediyl))bis(3-hydroxypropanoic acid). In still another example, the fluorescent tracer agent may be 3,6-Bis(2,5,8,11,14,17,20,23,26,29,32,35-dodecaoxaheptatriacontan-37-ylamino)-N2,N5-di(2,5,8,11,14,17,20,23,26,29,32,35-dodecaoxaheptatriacontan-37-yl)pyrazine-2,5-dicarboxamide. In a further example, the fluorescent tracer agent may be 3,6-Bis(2,5,8,11,14,17,20,23,26,29,32,35-dodecaoxaoctatriacontan-38-ylamino)-N²,N⁵-di(2,5,8,11,14,17,20,23,26,29,32,35-dodecaoxaheptatriacontan-37-yl)pyrazine-2,5-dicarboxamide. In yet another example, the fluorescent tracer agent may be D-serine, N,N′-[[3,6-bis[[[(2S)-2,3-dihydroxypropyl]amino]-2,5-pyrazinediyl]dicarbonyl]bis-, In an example, the fluorescent tracer agent may be 3,6-diamino-N²,N⁵-di(2,5,8,11,14,17,20,23,26,29,32,35,38,41,44,47,50,53,56,59,62,65,68,71-tetracosaoxatriheptacontan-73-yl)pyrazine-2,5-dicarboxamide. In another example, the fluorescent tracer agent may be 3,6-N,N′-Bis(2,3-dihydroxypropyl)-2,5-pyrazinedicarboxamide.

TABLE 1
Fluorescent Tracer Agents

| Tracer | Molecular | | |
|---|---|---|---|
| Agent | Weight | | |
| Name | (Da) | Structure | Chemical Name |
| MB- 102 | 372 | 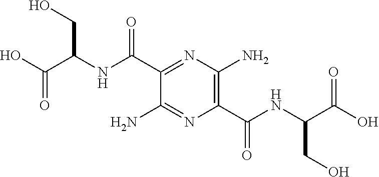 [SMILES: Nc1nc(C(=O)N[C@H](CO)C(=O)O)c(N)nc1C(=O)N[C@H](CO)C(=O)O] | 3,6-diamino-2,5-bis{N-[(1R)-1- carboxy-2- hydroxyethyl]carbamoyl}pyrazine; 3,6-diamino-N2,N5-bis(D- serine)-pyrazine-2,5- dicarboxamide |
| MB- 404 | 492 | 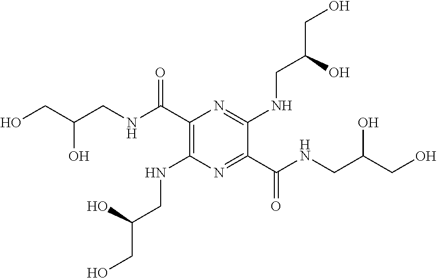 [SMILES: O=C(NCC(O)CO)c1nc(NC[C@H](O)CO)c(C(=O)NCC(O)CO)nc1NC[C@H](O)CO] | N²,N⁵-bis(2,3-dihydroxypropyl)- 3,6-bis[(S)-2,3- dihydroxypropylamino]pyrazine- 2,5-dicarboxamide |
| MB- 106 | 524 | 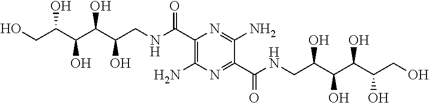 [SMILES: Nc1nc(C(=O)NC[C@@H](O)[C@H](O)[C@@H](O)[C@@H](O)CO)c(N)nc1C(=O)NC[C@@H](O)[C@H](O)[C@@H](O)[C@@H](O)CO] | 3,6-diamino-N²,N⁵- bis((2R,3S,4S,5S)-2,3,4,5,6- pentahydroxyhexyl)pyrazine-2-5- dicarboxamide |
| MB- 216 | 2367 | 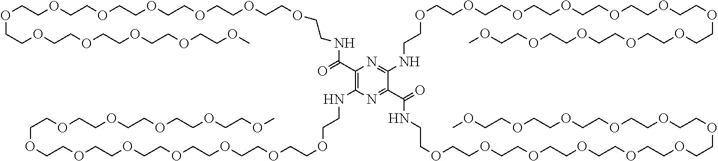 [SMILES: COCCOCCOCCOCCOCCOCCOCCOCCOCCOCCOCCOCCNC(=O)c1nc(NCCOCCOCCOCCOCCOCCOCCOCCOCCOCCOCCOCCOC)c(C(=O)NCCOCCOCCOCCOCCOCCOCCOCCOCCOCCOCCOCCOC)nc1NCCOCCOCCOCCOCCOCCOCCOCCOCCOCCOCCOCCOC] | 3,6- Bis(2,5,8,11,14,17,20,23,26,29, 32,35-dodecaoxaheptatriacontan- 37-ylamino)-N²,N⁵- di(2,5,8,11,14,17,20,23,26,29,32, 35-dodecaoxaheptatriacontan- 37-yl)pyrazine-2,5- dicarboxamide |
| MB- 212 | 2395 | 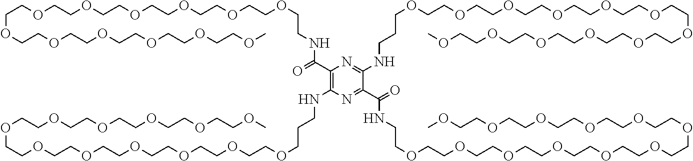 [SMILES: COCCOCCOCCOCCOCCOCCOCCOCCOCCOCCOCCOCCCNc1nc(C(=O)NCCOCCOCCOCCOCCOCCOCCOCCOCCOCCOCCOCCOC)c(NCCCOCCOCCOCCOCCOCCOCCOCCOCCOCCOCCOCCOC)nc1C(=O)NCCOCCOCCOCCOCCOCCOCCOCCOCCOCCOCCOCCOC] | 3,6- Bis(2,5,8,11,14,17,20,23,26,29, 32,35-dodecaoxaoctatriacontan- 38-ylamino)-N²,N⁵- di(2,5,8,11,14,17,20,23,26,29,32, 35-dodecaoxaheptatriacontan- 37-yl)pyrazine-2,5- dicarboxamide |
| MB- 116 | 2250 | 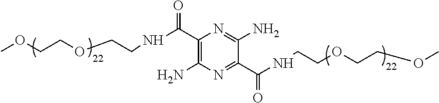 | 3,6-diamino-N²,N⁵- di(2,5,8,11,14,17,20,23,26,29,32, 35,38,41,44,47,50,53,56,59,62, 65,68-tricosaoxaheptacontan- 70-yl)pyrazine-2,5- dicarboxamide |
| MB- 206 | 520 | 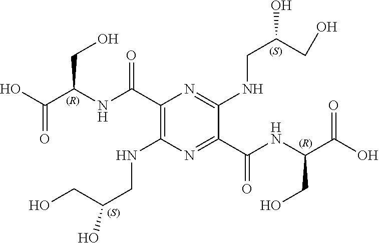 | D-Serine,N,N′-[[3,6-bis[[(2S)-2,3- dihydroxypropyl]amino]-2,5- pyrazinediyl]dicarbonyl]bis- |
| MB- 112 | 2339 | 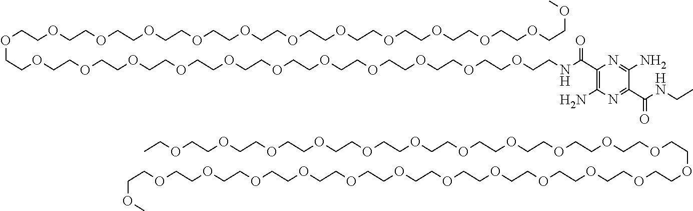 | 3,6-diamino-N²,N⁵- di(2,5,8,11,14,17,20,23,26,29,32, 35,38,41,44,47,50,53,56,59,62, 65,68,71- tetracosaoxatriheptacontan-73- yl)pyrazine-2,5-dicarboxamide |
| MB- 402 | 344 | 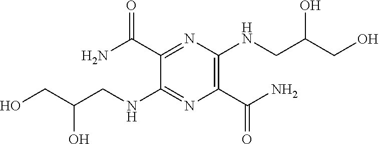 [SMILES: NC(=O)c1nc(NCC(O)CO)c(C(N)=O)nc1NCC(O)CO] | 3,6-N,N′-Bis(2,3- dihydroxypropyl)-2,5- pyrazinedicarboxamide |

The fluorescent tracer agent may be administered by any suitable method that delivers it to the gastrointestinal tract of the patient. Examples of enteral administration include, but are not limited to, oral, endoscopic instillation, sublingual, buccal and rectal routes, and delivery by swallowed diagnostic devices (i.e., Pill-Cam). Enteral dosage forms may include, but are not limited to liquid dosage forms such as a solution for enteral use, aqueous suspensions such as capsules, emulsions, gels, syrups, elixirs, magmas or mixtures thereof, or solid dosage forms such as powders, effervescent powders, capsules, tablets, effervescent tablets, extended release tablets and capsules. In one example, sublingual and buccal dosage forms may include tablets and lozenges. Rectal dosage forms may include suppositories, enemas and ointments. In one example, a bolus of the fluorescent tracer may be delivered at the most proximal point in the colon during a colonoscopy, through an endoscope, as a method to gain additional sensitivity to the visualization. In this example, organ health may be assessed and biopsies may be taken as further metrics of mucosal healing.

Detection and/or measurement of the fluorescent tracer may be achieved by optical fluorescence, absorbance, and/or light scattering methods using invasive and/or non-invasive probes such as optical sensors on the skin, in endoscopes, catheters, ear clips, hand bands, headbands, finger probes, and/or the like. Imaging can be achieved using planar imaging, optical tomography, optical coherence tomography, endoscopy, light scattering technology, laser assisted guided surgery, confocal microscopy, and/or light scattering devices. In an embodiment, the amount of the administered dose is measured in the patient's urine. The amount of fluorescent tracer in the patient's urine may be measured using an optical sensor and/or high performance liquid chromatography (HPLC). In another embodiment, fluorescence indicative of the amount of fluorescent tracer in the patient's blood and tissues may be measured using an optical transcutaneous sensor. The fluorescent tracer may be measured using any transcutaneous sensor operable to irradiate and detect the fluorescent tracer through intact skin, such as the transcutaneous sensors described in U.S. Pat. Nos. 10,765,354, 10,548,521, 10,264,977, and 10,194,854, 11,261,165, and US Patent Publication No. 20210369164 and 20220022820 and incorporated herein by reference. In an example, the transcutaneous sensor may irradiate the fluorescent agent in the patient's blood and tissues with non-ionizing radiation, causing the agent to fluoresce and detect the fluorescence of the fluorescent tracer agent. The non-ionizing radiation may be light in the near-infrared (NIR) or visible spectrum of about 350 nm or greater. In some examples, the non-ionizing radiation may have a wavelength of at least 350 nm to about 900 nm.

### EXAMPLES

### Example 1: Higher Ratio of Fluorophores Recovered in Urine of Rats with Small Bowel Injury

DSAT was mimicked using two fluorophores with similar molecular weights to mannitol and lactulose, MB-301 and MB-404 (see Table 2). Like lactulose, MB-404 is too large to be absorbed by a healthy gut, but can traverse intercellular spaces in a diseased gut exhibiting increased permeability.

TABLE 2
Fluorescent Tracer Agents

| Tracer Agent | Fluorescent tracer agents | | |
|---|---|---|---|
| Name | MB-301 | MB-102 | MB-404 |
| USAN | | relmapirazin | |
| Molecular | 198 | 372 | 492 |
| Weight (Da) | | | |
| Light | 405/540 | 445/560 | 500/620 |
| absorption/ | | | |
| emission | | | |
| spectra (nm) | | | |
| Structure | 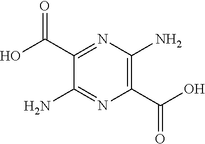 [SMILES: Nc1nc(C(=O)O)c(N)nc1C(=O)O] | 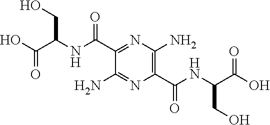 [SMILES: Nc1nc(C(=O)N[C@H](CO)C(=O)O)c(N)nc1C(=O)N[C@H](CO)C(=O)O] | 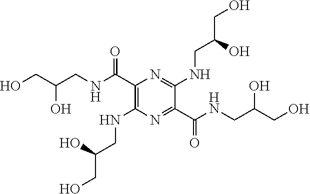 [SMILES: O=C(NCC(O)CO)c1nc(NC[C@H](O)CO)c(C(=O)NCC(O)CO)nc1NC[C@H](O)CO] |
| Scientific | 3,6-diamino-2,5- | 3,6-diamino-2,5-bis{N-[(1R)-1- | N²,N⁵-bis(2,3-dihydroxypropyl)- |
| name | pyrazine- | carboxy-2- | 3,6-bis[(S)-2,3- |
| | dicarboxylic acid | hydroxyethyl]carbamoyl}pyrazine | dihydroxypropylamino]pyrazine- |
| | | | 2,5-dicarboxamide |

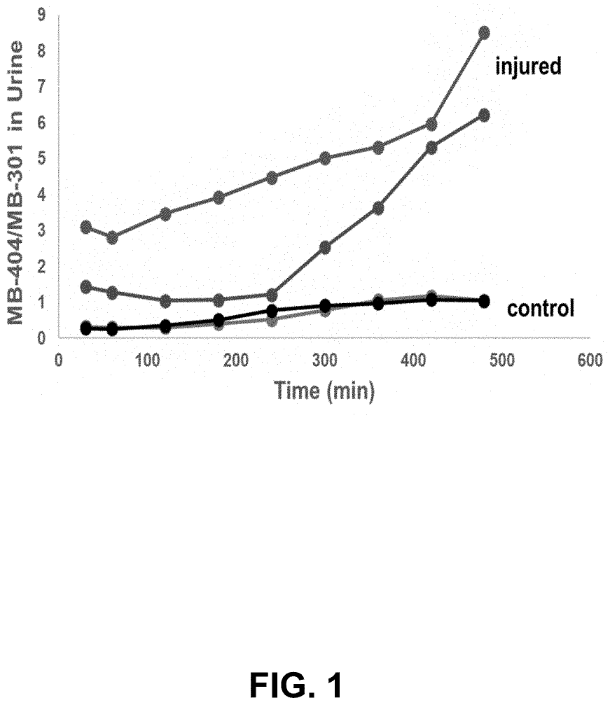

Furthermore, like both sugars used in the DSAT, MB-301 and MB-404 are not metabolized and are excreted by glomerular filtration, with neither (or negligible) tubular secretion nor reabsorption. To test the ability to distinguish between healthy and diseased gut, a rat model of small bowel injury was used with two injured and two control rats. The day after indomethacin-induced bowel injury, the rats were anaesthetized, a catheter was inserted for urine collection, and both fluorophores were delivered by oral gavage. Urine was collected prior to oral gavage and at a series of time points post-gavage. Concentrations of MB-301 and MB-404 in urine were determined using HPLC, and the ratio of their concentrations was plotted over time in FIG. 1.

As expected, the ratio of MB-404 to MB-301 in urine was higher in injured rats compared to control rats, reflecting increased gut permeability in the injured rats. Thus, use of MB-301 and MB-404 effectively mimicked the DSAT, with the ratio of their concentration in urine allowing distinction between healthy and diseased intestinal mucosa. In addition, the fluorescent tracers provided smoother curves, and can be measured without taking possession of the specimens if a transcutaneous detection system is used.

### Example 2: Transdermal Detection of High Molecular Weight Fluorophore in Rat Model of Bowel Injury

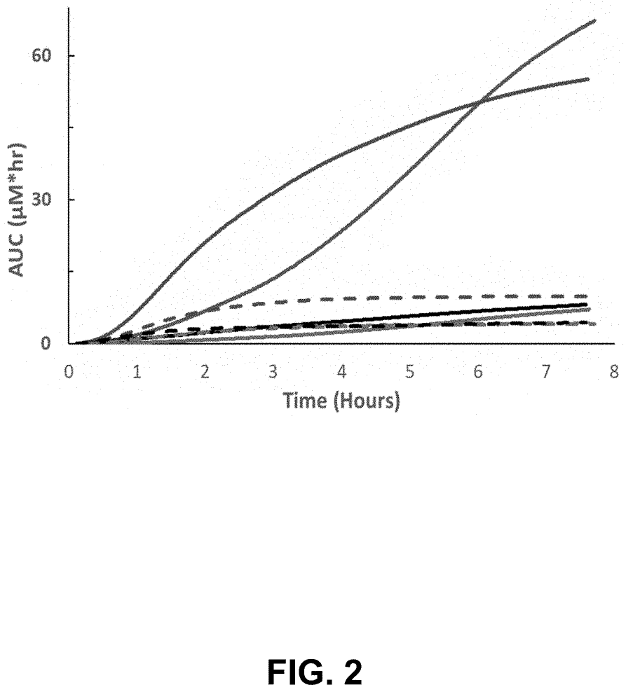

To probe the ability to assess gut function in a specimen-free and real-time manner, a dual-wavelength transdermal fluorescence detection system was utilized to monitor MB-301 and MB-404 in the rat model of bowel injury. As the excitation and emission spectra of these fluorophores do not overlap, their presence can be assayed simultaneously. Following anaesthetization, the fluorescence detection system was affixed with medical adhesive to a depilated area on the back of each rat. Transdermal fluorescence monitoring was then initiated to detect baseline fluorescence prior to delivery of MB-301 and MB-404 by oral gavage, and was monitored continuously. The concentration of MB-301 as determined by transdermal fluorescence measurements remained low in both injured and control animals (FIG. 2, dashed lines) as this fluorophore is comparatively rapidly absorbed by the small intestine and subsequently cleared. Similarly, the concentration of MB-404 in control animals also remained low throughout the course of the experiment, indicating that this fluorophore did not traverse the intestinal barrier and enter circulation. On the other hand, transdermal detection of MB-404 in injured animals increased over time, reflecting increased gut permeability in these animals. Therefore, transdermal detection of MB-301 and MB-404 fluorescence can be used to provide a non-invasive, real-time indication of gut health.

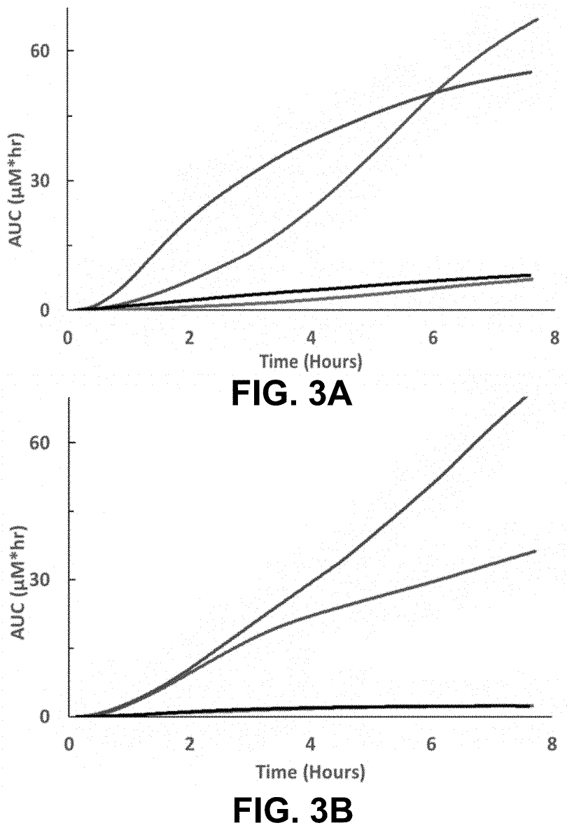

Further analysis of this data was done by looking at each component molecule result separately. Transdermal detection of MB-404 alone allowed for obvious delineation between healthy rats and those with bowel injury as shown in FIG. 3. Thus, a single, high molecular weight fluorophore can be used to assess gut permeability. This is advantageous for regulatory and commercial needs.

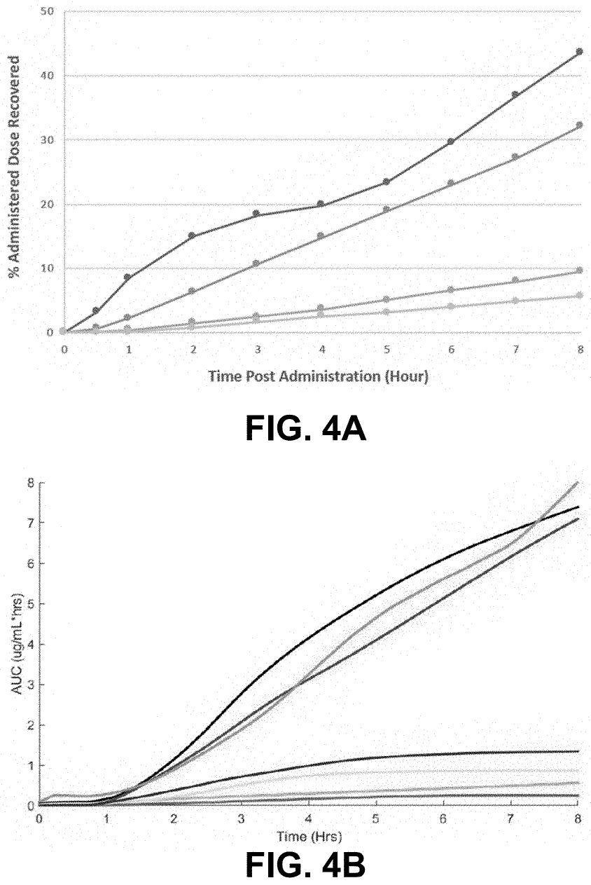

To this end, MB-102 was tested in the rat model of small bowel injury (FIGS. 4A and 4B). Transdermal measurements were initiated prior to oral gavage of MB-102 and transdermal measurements, alongside urine, were collected over time. Based on urine samples the Percent of Administered Dose Recovered was higher in rats with bowel injury than controls (FIG. 4A). As seen with MB-404 (FIGS. 3A and 3B), there was also clear delineation between injured rats and healthy controls in the transdermal fluorescence data (FIG. 4B), demonstrating that MB-102 can be used alone to assess gut permeability.

### Example 3: Quantification of Intestinal Permeability in Human Crohn's Disease

A single center, randomized, open label, crossover study was performed to evaluate a solution containing 18.6 mg/mL of a pyrazine derived fluorophore (MB-102) at 1.5 or 3.0 mg/kg versus DSAT (L, 1000 mg and R, 200 mg) administered enterally. The composition of MB-102 solution for use in this study is provided in Table 3.

TABLE 3
Composition of MB-102 solution for oral ingestion

| | | Quantity per mL of | |
|---|---|---|---|
| Component | Function | Formulation | |
| MB-102 | Active Ingredient | 18.824 | mg |
| Sodium dihydrogen phosphate | Diluent/Buffer | 2 | mg |
| monohydrate, BP, USP | | | |
| Sodium chloride, Ph. Eur | Diluent/Buffer | 4.62 | mg |

| | | |
|---|---|---|
| Water for injection, Ph. Eur | Diluent/Buffer | To 1014 mg |
| 1N Sodium hydroxide | Diluent/Buffer | pH adjusted to |
| | | 7.0-7.4 |

To compare the dynamic ranges of the fluorophore and DSAT clearances, healthy adults and adults with evidence of small bowel Crohn's disease on magnetic resonance enterography were enrolled. Participants were randomized to receive either the fluorophore tracer or the sugars on treatment day 1, followed by the other tracer 3-7 days later. Urine was collected at baseline and 1, 2, 4, 6, 8, 10, and 12 hours after tracer ingestion. The cumulative urinary percent recovery of the fluorophore tracer was calculated using an Ultra-Performance Liquid Chromatography system with fluorescence detection and cumulative L, R, and L:R ratios using a normal phase, isocratic HPLC-tandem ESI mass spectrometry.

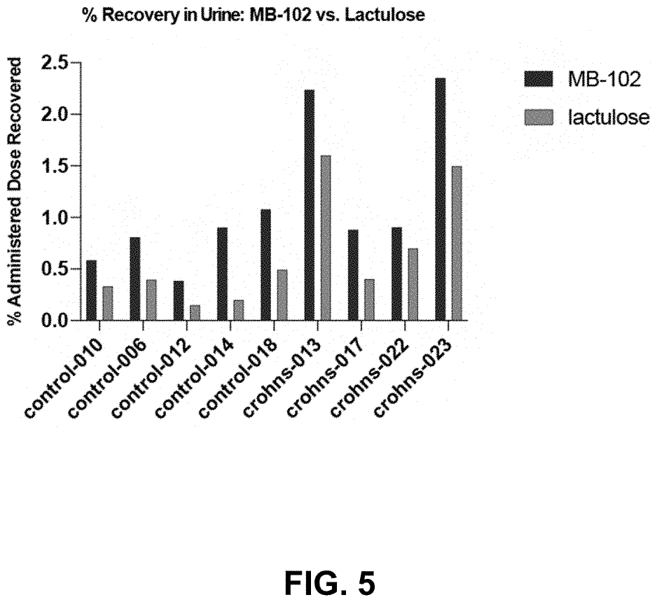

5 healthy adults and 4 adults with Crohn's disease completed the study without serious adverse events. As shown in FIG. 5, percent recovery of the fluorophore tracer (MB-102) in urine correlates with percent lactulose recovered in the urine for all participants (r=0.867, p=0.005). In addition, the percent lactulose or MB-102 recovered in controls is similar across participants. At 12 hours post-dose in the healthy participants, approximately 0.5% to 1.0% of the administered dose was recovered, reflecting the inability of MB-102 to transverse the intestinal barrier when gut permeability is normal. In contrast, Crohn's disease patients have a wide range of percent lactulose or MB-102 recovered, which likely reflects the variable degree of disease control at the time of testing.

In addition, two healthy participants and one participant with Crohn's disease were administered an oral dose of MB-102, and blood samples were collected at 6 and 12 hours post-administration. MB-102 plasma concentration levels were determined by HPLC. These data demonstrate that increased excretion of MB-102 dose and elevated MB-102 plasma concentration can be seen between 6-12 hours post-administration in Crohn's disease (Table 3).

TABLE 3
Detection of MB-102 in plasma concentration
of healthy and diseased participants

| | Plasma Concentration (ng/mL) | | |
|---|---|---|---|
| | | | Crohn's Disease |
| Time (min.) | | Control Participants | Participant |

| | | | |
|---|---|---|---|
| 360 | 21.2 | 21.3 | 63.1 |
| 720 | 16.79 | 16.7 | 26.5 |

MB-102 is detected in urine in a small bowel injury animal model and can distinguish between bowel injury and control as measured by % dose recovered over time (FIG. 4A). In addition, transdermal fluorescence detection of MB-102 in this same animal model can also distinguish between bowel injury and control (FIG. 4B). In translation to humans, oral administration of MB-102 results in increased urine excretion in diseased participants with respect to controls (FIG. 5).

Orally administered MB-102 caused no serious adverse events and was well tolerated. Importantly, excretion of percent lactulose and MB-102 are highly correlated across a wide range of values. Patients that were in remission when study was performed showed near normal values for MB-102 urine recovery, which presumably can be interpreted as mucosal healing after treatment.

### Example 4: Measurement of Mucosal Healing in Colitis

In colitis, colonic permeability is increased. During a colonoscopy, the endoscopist may deliver a bolus of MB-102 at the most proximal point in the colon, through the scope, as a method to gain additional sensitivity to the visualization and the biopsies as metrics of mucosal healing. In this example, there may be transcutaneous measurement of uptake into the patient's system starting at the time of delivery. In some instances, the fluorophore may be in a gel, so that the liquid doesn't obscure the field of vision of the colonoscope on withdrawal. Additionally, at the first (diagnostic) colonoscopy, the endoscopist may deliver a payload of MB-102 as a “baseline” pre-treatment measurement of organ permeability.

In ulcerative colitis, there is an axial gradient, such that the disease is always worst most distally (or, in severe cases, it is equally abnormal throughout the colon, so called pan-colitis). Hence, the distal part of the colon is the region that is last to improve. A smaller volume instillation of MB-102 into the rectum may provide relatively quicker (short duration of tracing) assessment of mucosal integrity. There is value to total colonoscopy for cancer surveillance in ulcerative colitis, but using the fluorescent tracer may allow for limiting total colonoscopies to once every year or two, with interim assessment of disease control being generated by rectal installation of the fluorophore(s).

### Example 5: Measurement of Mucosal Healing in Graft Vs. Host Disease (GVHD)

GVHD of the intestines is a major cause of morbidity and mortality following bone marrow/stem cell transplant. In this disorder, the grafted (donor) cells attack the recipient's organs. Treatment is via increased immune suppression. The duration of treatment is guided empirically—when the symptoms improve, after some interval, the immune suppression is lessened.

MB-102 may be administered and then measured to predict the onset of GVHD based on an increase in the amount of the fluorescent tracer recovered/measured after a bone marrow/stem cell transplant. After the onset of GVHD, the use of MB-102 may be used to indicate resolution (e.g., mucosal healing). Once mucosal healing is determined based on a reduction in the amount of the fluorescent tracer recovered/measured, the immune suppression may be lessened.

### Example 6: Measurement of Mucosal Healing in IBD

A patient with inflammatory bowel disease, such as Crohn's Disease, of variable duration needs confirmation that his/her mucosa is well healed. The criteria traditionally used to determine improvement since the last change in therapy (from biologic A to biologic B) are laboratory based (circulating concentration of C-reactive protein, a marker of inflammation, and fecal calprotectin). If these values have diminished on the current therapy, the patient has, by many criteria, greatly improved. However, the fecal calprotectin is not a perfect test, because it is prone to sampling error within a specimen, and time of collection influence, and degradation in transport.

If the patient's Harvey-Bradshaw index is greatly improved since the last change in/start of medication, but they report having mild abdominal pain on many days of the week, and stools remain loose, the doctor and patient may be reasonably pleased with the outcome. The patient may also have returned to work, have increased weight and appetite, and not have side effects to the biologic. Previously, this improvement in objective values (CRP, fecal calprotectin, weight) and subjective assessments (the Harvey-Bradshaw Index, improved appetite) would offer confidence that one can maintain the treatment. However, there could always be some room to maneuver, such as increasing the dose of the biologic, giving it more frequently, adding adjunctive treatment, or even switching to a different biologic. This depends on the best possible technique to make sure that the mucosa is actually healed.

In this case, the doctor wants to understand if the mild symptoms reflect an incompletely healed gut. Even if the patient were symptom free, in view of the high desirability of having complete mucosal healing, the patient would likely undergo an endoscopic assessment to view the lining of the intestines (the mucosa). Currently, there is little data as to whether taking biopsies adds to the visual assessment. If the mucosae (colon, distal small bowel) appear normal, then this might be the limits of the assessment. If all appears well, generally treatment wouldn't be escalated. However, there is risk to this strategy, because the patient could still have active disease proximal to the colonoscope's reach, and even with normal appearance there might be scattered defects in permeability.

Therefore, MB-102 may be administered and the amount recovered/measured outside the gut may be used to assess mucosal healing. There are no such pan-intestinal assessments (the mucosal appearance is prone to insensitivity as noted immediately above). Finding reversion of the permeability towards normal (a range as defined by a database (a reference range)) may be used initially to assess that the timing is right to assess mucosal healing endoscopically or colonoscopically. Alternatively, the permeability defect might be related to baseline at the start of treatment, or when the biologic had been switched. If the permeability hasn't returned to normal, it may mean the continued treatment is warranted before the colonoscopy, or possibly there is a need for increased immune suppression. The time frame between endoscopic procedures is about 9-12 months, so more rapid information gleaned from the permeability testing may offer considerable interim value.

### Example 7: Measurement of Gut Permeability in Extra-Intestinal Disease

At the time of an event (e.g. types 1 and 2 diabetes, nonalcoholic fatty liver disease, and certainly inflammatory bowel disease), intestinal permeability is increased. For the first three of these disorders, there are some data that increased intestinal permeability precedes their onset. That sequence of events suggests that increased gut permeability may contribute to the damage to the pancreas and liver, or to insulin resistance. Patients may be screened to detect this defect in barrier function (in conjunction with more standard assessments such as hemoglobin A1C, fasting blood sugar, liver enzyme tests), and as these blood tests begin to embark from baseline, or even before these surrogate tests become abnormal, permeability may be a either a target for treatment (gut microbiome manipulation, diet, pharmaceuticals), or indicate more assiduous and frequent screening.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should be interpreted as illustrative and not be taken as limiting the scope of the invention. The following claims are intended to cover all generic and specific features described herein, as well as all statements of the scope of the present method, which, as a matter of language, might be said to fall there between.

What is claimed is:

1. A method for monitoring mucosal healing in a patient with a digestive disease or in a pre-disease state, the method comprising:
establishing a baseline of the patient comprising:
enterally administering a first dosage of a composition comprising a fluorescent tracer that is not substantially absorbed by a healthy gut;
measuring, via fluorescence, a first amount of the administered dose that can be found outside the gut over a period of time; and
determining a baseline total percentage of the administered dose recovered,
treating the patient for the digestive disease or the pre-disease state; measuring gut permeability of the patient after treatment comprising:
enterally administering a second dosage of the composition comprising the fluorescent tracer;
measuring, via fluorescence, a second amount of the administered dose that can be found outside the gut over a period of time; and
determining a second total percentage of the administered dose recovered; and
comparing the second total percentage of the administered dose recovered to the baseline total percentage of the administered dose recovered.

2. The method of claim 1, wherein the first and second amount of the administered dose is measured in the patient's urine.

3. The method of claim 1, wherein the first and second amount of the administered dose is measured transdermally, via a transdermal sensor, wherein the transdermal sensor:
irradiates the composition in the patient's blood and/or tissue with non-ionizing radiation, causing the composition to fluoresce; and
detects the fluorescence of the fluorescent tracer in the patient's blood and/or tissue.

4. The method of claim 1 further comprising assessing the patient's mucosal healing based on the comparison of the second total percentage of the administered dose recovered to the baseline total percentage of the administered dose recovered and to a distribution of values the administered dose recovered in a normal population.

5. The method of claim 4, wherein when the second total percentage of the administered dose recovered is less than the baseline total percentage of the administered dose recovered and less than or equal to two standard deviations above the mean of the recovered administered dose in a normal population, the patient likely has mucosal healing.

6. The method of claim 4, wherein when the second total percentage of the administered dose recovered is less than the baseline total percentage of the administered dose recovered but remains above 2 standard deviations above the mean of the recovered administered dose in a normal population, the patient likely does not have mucosal healing.

7. The method of claim 6 further comprising selecting an intervention for the patient and assessing mucosal healing after the intervention.

8. The method of claim 1, wherein the mucosal healing is assessed after treatment for at least 1 week.

9. The method of claim 1, wherein the digestive disease is selected from the group consisting of Crohn's disease, ulcerative colitis, celiac disease, and graft-versus-host disease.

10. The method of claim 1, wherein the first dosage and the second dosage are about 1.5 mg/kg to about 50 mg/kg.

11. The method of claim 1, wherein the fluorescent tracer comprises a pyrazine.

12. The method of claim 11, wherein the pyrazine is 3,6-diamino-2,5-bis{N-[(1R)-1-carboxy-2-hydroxyethyl]carbamoyl}pyrazine or 3,6-diamino-N2,N5-bis(D-serine)-pyrazine-2,5-dicarboxamide.

13. A method for determining mucosal healing in a patient with a digestive disease, the method comprising:
enterally administering a dosage of a composition comprising a fluorescent tracer that is not substantially absorbed by a healthy gut;
measuring, via fluorescence, an amount of the administered dose that can be found outside the gut over a period of time; and
determining a total percentage of the administered dose recovered, wherein the total percentage of the administered dose recovered correlates to the patient's mucosal healing.

14. The method of claim 13, wherein the amount of the administered dose is measured in the patient's urine.

15. The method of claim 13, wherein the amount of the administered dose is measured transcutaneously, via a transcutaneous sensor, wherein the transcutaneous sensor:
irradiates the composition in the patient's blood and/or tissue with non-ionizing radiation, causing the composition to fluoresce; and detects the fluorescence of the fluorescent tracer in the patient's blood and/or tissue.

16. The method of claim 13 further comprising assessing the patient's mucosal healing.

17. The method of claim 16, wherein the patient likely has mucosal healing if the total percentage of the administered dose recovered is less than or equal to 1.5%.

18. The method of claim 16, wherein the patient likely does not have mucosal healing if the total percentage of the administered dose recovered is greater than 2%.

19. The method of claim 16, wherein the patient's mucosal healing is assessed by comparing the total percentage of the administered dose recovered to a distribution of values of the administered dose recovered in a normal population.

20. The method of claim 19, wherein if the total percentage of the administered dose recovered is less than or equal to two standard deviations above the mean of the recovery of the administered dose in a normal population, the patient likely has mucosal healing.

21. The method of claim 19, wherein if the total percentage of the administered dose recovered is greater than two standard deviations above the mean of the recovery of the administered dose in a normal population, the patient likely does not have mucosal healing.

22. The method of claim 13, wherein the digestive disease is selected from the group consisting of Crohn's disease, ulcerative colitis, celiac disease, and graft-versus-host disease.

23. The method of claim 13, wherein the dosage is about 1.5 mg/kg to about 50 mg/kg.

24. The method of claim 13, wherein the fluorescent tracer comprises a pyrazine.

25. The method of claim 24, wherein the pyrazine is 3,6-diamino-2,5-bis{N-[(1R)-1-carboxy-2-hydroxyethyl]carbamoyl}pyrazine or 3,6-diamino-N2,N5-bis(D-serine)-pyrazine-2,5-dicarboxamide.
